(19) United States
(12) Patent Application Publication
(10) Pub. No.: US 2018/0243155 A1
(43) Pub. Date: Aug. 30, 2018

(54) Systems and Methods for Creating Custom-Fit Exoskeletons

(71) Applicant: Ekso Bionics, Inc., Richmond, CA (US)

(72) Inventors: Russ ANGOLD, American Canyon, CA (US); Adam PREUSS, Santa Rosa, CA (US); Nicholas FLEMING, San Francisco, CA (US); Kurt AMUNDSON, Berkeley, CA (US)

(73) Assignee: Ekso Bionics, Inc., Richmond, CA (US)

(21) Appl. No.: 15/758,148

(22) PCT Filed: Sep. 9, 2015
(86) PCT No.: PCT/US15/49169

§ 371 Date: Mar. 7, 2018

(51) Int. Cl. A61H 3/00; G01B 11/24; G06T 17/00; G06T 19/20; B33Y 50/00; B33Y 80/00

(52) U.S. Cl. CPC A61H 3/00; G01B 11/24; G06T 17/00; A61B 5/1079; B33Y 50/00; B33Y 80/00; G06T 19/20

(57) ABSTRACT

A three-dimensional surface scan of an exoskeleton wearer is performed to generate three-dimensional surface data, and a three-dimensional surface model of the exoskeleton wearer is generated from the three-dimensional surface scan data. A three-dimensional exoskeleton model is generated from the three-dimensional surface model. At least one three-dimensional exoskeleton component is printed from the three-dimensional exoskeleton model, and a custom-fit exoskeleton is assembled using the at least one three-dimensional exoskeleton component.

### FIELD OF THE INVENTION

The present invention relates to devices and methods that augment a user's strength or aid in the prevention of injury during the performance of certain motions or tasks. More particularly, the present invention relates to devices and methods suitable for use by a person engaging in heavy tool use or weight bearing tasks or to devices and methods suitable for therapeutic use with patients that have impaired neuromuscular or muscular function of the appendages. These devices comprise a set of artificial limbs, and in some cases related control systems and actuators, that potentiate improved function of the user's appendages for activities including, but not limited to, enabling walking for a disabled person, granting greater strength and endurance in a user's arms or allowing for more weight to be carried by the user while walking.

### BACKGROUND OF THE INVENTION

Wearable exoskeletons have been designed for medical, commercial and military applications. Medical exoskeletons are used to restore and rehabilitate proper muscle function for people with disorders that affect muscle control. Medical exoskeletons include a system of motorized braces that can apply forces to a user's appendages. In a rehabilitation setting, medical exoskeletons are controlled by a physical therapist who uses one of a plurality of possible input means to command an exoskeleton control system. In turn, the medical exoskeleton control system actuates the position of the motorized braces, resulting in the application of force to, and typically movement of, the body of the exoskeleton user. Commercial and military exoskeletons help prevent injury and augment an exoskeleton user's stamina and strength by alleviating loads supported by workers or soldiers during their labor or other activities. Tool holding commercial exoskeletons are outfitted with a tool holding arm that supports the weight of a tool, thereby reducing user fatigue by providing tool holding assistance. The tool holding arm transfers the vertical force required to hold the tool through the legs of the exoskeleton rather than through the user's arms. Similarly, military weight bearing exoskeletons transfer the weight of a load, such as armor or a heavy backpack, through the legs of the exoskeleton rather than through the user's legs. Commercial and military exoskeletons can have actuated joints that augment the strength of the exoskeleton user, with these actuated joints being controlled by an exoskeleton control system and with the exoskeleton user using any of a plurality of possible input means to command the exoskeleton control system.

In powered exoskeletons, exoskeleton control systems prescribe and control trajectories in the joints of the exoskeleton, which results in movement of the exoskeleton. These control trajectories can be prescribed as position-based, force-based or a combination of both methodologies, such as that seen in an impedance controller. Position-based control systems can be modified directly through modification of the prescribed positions. Force-based control systems can also be modified directly through modification of the prescribed force profiles. Complicated exoskeleton movements, such as walking in an ambulatory medical exoskeleton, are commanded by an exoskeleton control system through the use of a series of exoskeleton trajectories, with increasingly complicated exoskeleton movements requiring an increasingly complicated series of exoskeleton trajectories. These series of trajectories can be cyclic, such as the exoskeleton taking a series of steps with each leg, or they may be discrete, such as an exoskeleton rising from a seated position into a standing position. In the case of an ambulatory exoskeleton, during a rehabilitation session or over the course of rehabilitation, it is highly beneficial for the physical therapist to have the ability to modify the prescribed positions or the prescribed force profiles depending on the particular physiology or rehabilitation stage of a patient. It is highly complex and difficult to construct an exoskeleton control interface that enables the full range of modification desired by a physical therapist during rehabilitation. In addition, it is important that the control interface not only allow the full range of modifications that may be desired by the physical therapist, but that the interface with the physical therapist be intuitive to the physical therapist, who may not be highly technically oriented. As various exoskeleton users may be differently proportioned, variously adjusted or customized powered exoskeletons will fit each user somewhat differently, requiring that the exoskeleton control system take into account these differences in wearer proportion, exoskeleton configuration or customization and exoskeleton-user fit, which results in changes to the prescribed exoskeleton trajectories.

Regardless of the specific type of exoskeleton, the proper fit and sizing of an exoskeleton to an exoskeleton user increases the utility of the exoskeleton to the user. However, the proportions of people are highly variable, thereby complicating the proper fitting of an exoskeleton. In the case of an adjustable exoskeleton, a skilled technician or physical therapist is required to fit the exoskeleton to a specific user. Still, even with a well-designed adjustable exoskeleton and a skilled technician, the fit to a specific user may not be optimal in some cases. A better fit can be achieved through the custom manufacture of all or part of an exoskeleton for each specific user. However, the adoption of custom-manufactured exoskeleton parts using current methods is limited by the cost of personalized manufacture, the skillsets required for custom exoskeleton design and the time lag between measurement or fitting of a user and delivery of the custom parts.

Accordingly, there exists a need in the art for the ability to the simply, rapidly and accurately measure an exoskeleton user in order to allow for the subsequent design and manufacture of a personalized exoskeleton fitted to the specific user. It would be of additional utility if this measurement, design and manufacture could take place in the absence of highly skilled medical or exoskeleton design personnel. It would be of further utility if this measurement, design and manufacture could take place in locations other than at a specific exoskeleton manufacturing company, such as in theatre for military exoskeletons or in hospital or clinical environments for medical exoskeletons. There additionally exists a need to provide for the modeling of exoskeleton and user movements for such personalized exoskeletons in order to allow for the subsequent alteration of trajectories prescribed by an exoskeleton control system of a personalized exoskeleton.

### SUMMARY OF THE INVENTION

It is an object of the present invention to provide a device and method that allows for a rapid three-dimensional (3D) surface measurement of a person, modeling of the 3D surface of the measured person, design of personalized exoskeleton parts to best fit the measured person and manufacture of these personalized exoskeleton parts. It is an additional object of the present invention to provide a device and method that allows for a rapid 3D surface measurement of a person in multiple poses, modeling the 3D surface of the measured person in multiple poses, creation of a unified 3D surface model of the person measured, design of personalized exoskeleton parts to best fit the measured person and manufacture of these personalized exoskeleton parts.

It is an additional object of the present invention to provide a device and method that allows for a rapid 3D surface measurement and modeling of a person, the subsurface measurement and modeling of a person, creation of a unified surface and subsurface model of the person, design of personalized exoskeleton parts to best fit the measured person and manufacture of these personalized exoskeleton parts. It is an additional object of the present invention to provide a device and method that allows for a rapid surface and/or subsurface measurement and modeling of a person, design of personalized powered exoskeleton parts to best fit the measured person, creation of a unified model of the person and the personalized powered exoskeleton, generation of modified exoskeleton trajectories based on this unified model and upload of the modified trajectories to the exoskeleton control system of the personalized powered exoskeleton.

Concepts were developed for ways by which a physical therapist, technician or another person involved in the process of measuring the size of an exoskeleton user and manufacturing a personalized exoskeleton sized to fit that specific exoskeleton user can make use of 3D surface scanning devices to measure the surface dimensions and contours of the exoskeleton user. A computer is then used to model the 3D surface scan data to build a 3D surface model of the exoskeleton user. 3D computer modeling is used to design exoskeleton parts to optimally fit the 3D surface model of the exoskeleton user, and 3D printing is used to manufacture exoskeleton parts that will optimally fit the exoskeleton user, at which point a personalized exoskeleton can be assembled and fitted to the exoskeleton user using the custom-made exoskeleton parts.

Concepts were further developed for ways by which a physical therapist, technician or another person involved in the process of measuring the size of an exoskeleton user and manufacturing a personalized exoskeleton sized to fit that specific exoskeleton user can make use of 3D surface scanning devices to repeatedly measure the surface dimensions and contours of the exoskeleton user in various poses. A computer is then used to model the 3D surface scan data of the exoskeleton user in various poses to build a 3D surface model of the exoskeleton user in various poses and/or create a moving model of the exoskeleton user. 3D computer modeling is used to design exoskeleton parts to optimally fit the 3D surface model of the exoskeleton user, and 3D printing is used to manufacture exoskeleton parts that will optimally fit the exoskeleton user, at which point a personalized exoskeleton can be assembled and fitted to the exoskeleton user using the custom-made exoskeleton parts.

Concepts were further developed for ways by which a physical therapist, technician or another person involved in the process of measuring the size of an exoskeleton user and manufacturing a personalized exoskeleton sized to fit that specific exoskeleton user can make use of 3D surface scanning devices to measure the surface dimensions and contours of the exoskeleton user in one or more poses, followed by a second type of scan that measures the subsurface features of the exoskeleton user. A computer is then used to model the 3D surface scan data and subsurface scan data to build 3D surface and subsurface models of the exoskeleton user and/or create a moving model of the exoskeleton user. 3D computer modeling is used to design exoskeleton parts to optimally fit the 3D surface and subsurface models of the exoskeleton user, and 3D printing is used to manufacture exoskeleton parts that will optimally fit the exoskeleton user, at which point a personalized exoskeleton can be assembled and fitted to the exoskeleton user using the custom-made exoskeleton parts.

Concepts were developed for ways by which a physical therapist, technician or another person involved in the process of fitting a powered exoskeleton user and adjusting the trajectories of a personalized powered exoskeleton sized to fit that specific exoskeleton can make use of 3D surface scanning devices to measure the surface dimensions and contours of the exoskeleton user. A computer is then used to model the 3D surface scan data to build a 3D surface model of the exoskeleton wearer. 3D computer modeling is used to design exoskeleton parts to optimally fit the 3D surface model of the exoskeleton user, and 3D computer modeling is used to generate modified trajectories to control the personalized powered exoskeleton, at which point these modified trajectories are uploaded to the exoskeleton control system of the personalized powered exoskeleton.

Concepts were further developed for ways by which a physical therapist, technician or another person involved in the process of fitting a powered exoskeleton user and adjusting the trajectories of a personalized powered exoskeleton sized to fit that specific exoskeleton user can make use of 3D surface scanning devices to repeatedly measure the surface dimensions and contours of the exoskeleton user in various poses. A computer is then used to model the 3D surface scan data to build a 3D surface model of the exoskeleton user in various poses and/or create a moving model of the exoskeleton user. 3D computer modeling is used to design exoskeleton parts to optimally fit the 3D surface model of the exoskeleton user, and 3D computer modeling is used to generate modified trajectories to control the personalized powered exoskeleton and user, at which point these modified trajectories are uploaded to the exoskeleton control system of the personalized powered exoskeleton.

Concepts were further developed for ways by which a physical therapist, technician or another person involved in the process of fitting of a powered exoskeleton user and adjusting the trajectories of a personalized powered exoskeleton sized to fit that specific exoskeleton user can make use of 3D surface scanning devices to measure the surface dimensions and contours of the exoskeleton user in one or more poses, followed by a second type of scan which measures the subsurface features of an exoskeleton user. A computer is then used to model the 3D surface scan data and subsurface scan data to build 3D surface and subsurface models of the exoskeleton wearer and/or create a moving model of the exoskeleton wearer. 3D computer modeling is used to design exoskeleton parts to optimally fit the 3D surface and subsurface models of the exoskeleton user, and 3D computer modeling is used to generate modified trajectories to control the personalized powered exoskeleton and user, at which point these modified trajectories are uploaded to the exoskeleton control system of the personalized powered exoskeleton.

In particular, the present invention is directed to systems and methods for creating a custom-fit exoskeleton. A three-dimensional surface scan of an exoskeleton wearer is performed to generate three-dimensional surface data, and a three-dimensional surface model of the exoskeleton wearer is generated from the three-dimensional surface scan data. A three-dimensional exoskeleton model is generated from the three-dimensional surface model. At least one three-dimensional exoskeleton component is printed from the three-dimensional exoskeleton model, and the custom-fit exoskeleton is assembled using the at least one three-dimensional exoskeleton component.

In one embodiment, generating the three-dimensional surface model includes estimating a position of at least one joint of the exoskeleton wearer. The three-dimensional exoskeleton model is generated using the position of the at least one joint.

In another embodiment, a three-dimensional surface scan of the exoskeleton wearer is performed for each of a plurality of poses, and a three-dimensional surface model of the exoskeleton wearer is generated for each of the plurality of poses. The three-dimensional surface models are compiled into a unified three-dimensional surface model of the exoskeleton wearer. The three-dimensional exoskeleton model is generated from the unified three-dimensional surface model.

In still another embodiment, a subsurface scan of the exoskeleton wearer is performed to generate subsurface scan data, and a subsurface model of the exoskeleton wearer is generated from the subsurface scan data. The three-dimensional surface model and the subsurface model are compiled into a unified model. The three-dimensional exoskeleton model is generated from the unified model.

In yet another embodiment, a unified model is generated from the three-dimensional surface model and the three-dimensional exoskeleton model. At least one modified exoskeleton trajectory is generated using the unified model, and the at least one modified exoskeleton trajectory is uploaded to an exoskeleton control system of the custom-fit exoskeleton.

Additional objects, features and advantages of the invention will become more readily apparent from the following detailed description of the invention when taken in conjunction with the drawings wherein like reference numerals refer to corresponding parts in the several views.

### BRIEF DESCRIPTION OF THE FIGURES

FIG. 1 is a side view of a user wearing an ambulatory exoskeleton;

FIG. 2A is a front view of a soldier wearing a military exoskeleton;

FIG. 2B is a rear view of the soldier and exoskeleton;

FIG. 2C is a front view of the soldier wearing the military exoskeleton;

FIG. 2D is a partial cutaway view of the soldier and military exoskeleton, showing both the armor and the exoskeleton upon which the armor is mounted;

FIG. 3A is a flowchart illustrating a first embodiment of the present invention;

FIG. 3B shows a 3D surface scan of a person;

FIG. 3C is a front view of an exoskeleton user model generated from the 3D surface scan;

FIG. 3D is a rear view of the exoskeleton user model;

FIG. 3E is a front view of the exoskeleton user model with a model of customized exoskeleton parts superimposed over the exoskeleton user model;

FIG. 3F is a rear view of the exoskeleton user model and the model of customized exoskeleton parts;

FIG. 3G is a front view of a lower leg brace, of the model of customized exoskeleton parts, coupled to a lower right leg of the exoskeleton user model;

FIG. 3H is a rear view of the lower leg brace;

FIG. 3I is a perspective view of an exoskeleton constructed in accordance with the first embodiment;

FIG. 4A is a flowchart illustrating a second embodiment;

FIG. 4B shows a 3D surface scan of a person in a first pose;

FIG. 4C shows a 3D surface scan of the person in a second pose;

FIG. 4D is a front view of an exoskeleton user model generated from the 3D surface scan of the person in the first pose;

FIG. 4E is a front view of an exoskeleton wearer model generated from the 3D surface scan of the person in a different pose than that shown in FIG. 4D;

FIG. 5A is a flowchart illustrating a third embodiment;

FIG. 5B shows 3D surface and subsurface scans of a person;

FIG. 5C shows surface and subsurface models of the person;

FIG. 6 is a flowchart illustrating a fourth embodiment;

FIG. 7 is a flowchart illustrating a fifth embodiment; and

FIG. 8 is a flowchart illustrating a sixth embodiment.

### DETAILED DESCRIPTION OF THE PREFERRED EMBODIMENTS

Detailed embodiments of the present invention are disclosed herein. However, it is to be understood that the disclosed embodiments are merely exemplary of the invention that may be embodied in various and alternative forms. The figures are not necessarily to scale, and some features may be exaggerated or minimized to show details of particular components. Therefore, specific structural and functional details disclosed herein are not to be interpreted as limiting, but merely as a representative basis for teaching one skilled in the art to employ the present invention.

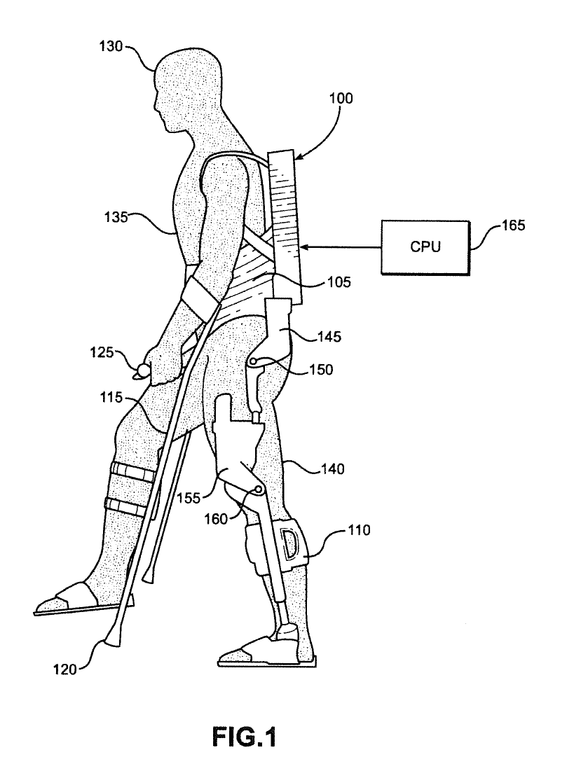

With reference to FIG. 1, an exoskeleton (or exoskeleton device) 100 has a torso portion 105 and leg supports (one of which is labeled 110). Exoskeleton 100 is used in combination with a pair of crutches, a left crutch 115 of which includes a lower, ground engaging tip 120 and a handle 125. In connection with this embodiment, through the use of exoskeleton 100, a patient (or, more generally, a user or wearer) 130 is able to walk. In a manner known in the art, torso portion 105 is configured to be coupled to a torso 135 of patient 130, while the leg supports are configured to be coupled to the lower limbs (one of which is labeled 140) of patient 130. Additionally, actuators, interposed between portions of the leg supports 110, as well as between the leg supports 110 and torso portion 105, are provided for shifting of the leg supports 110 relative to torso portion 105 to enable movement of the lower limbs 140 of patient 130. In some embodiments, torso portion 105 can be quite small and comprise a pelvic link (not shown), which wraps around the pelvis of patient 130. In the example shown in FIG. 1, the actuators are specifically shown as a hip actuator 145, which is used to move a hip joint 150 in flexion and extension, and as knee actuator 155, which is used to move a knee joint 160 in flexion and extension. The actuators 145 and 155 are controlled by a controller (or CPU) 165 in a plurality of ways known to one skilled in the art of exoskeleton control, with controller 165 being a constituent of an exoskeleton control system. Although not shown in FIG. 1, various sensors are in communication with controller 165 so that controller 165 can monitor the orientation of exoskeleton 100. Such sensors can include, without restriction, encoders, potentiometers, accelerometer and gyroscopes, for example. As certain particular structure of an exoskeleton for use in connection with the present invention can take various forms and is known in the art, it will not be detailed further herein.

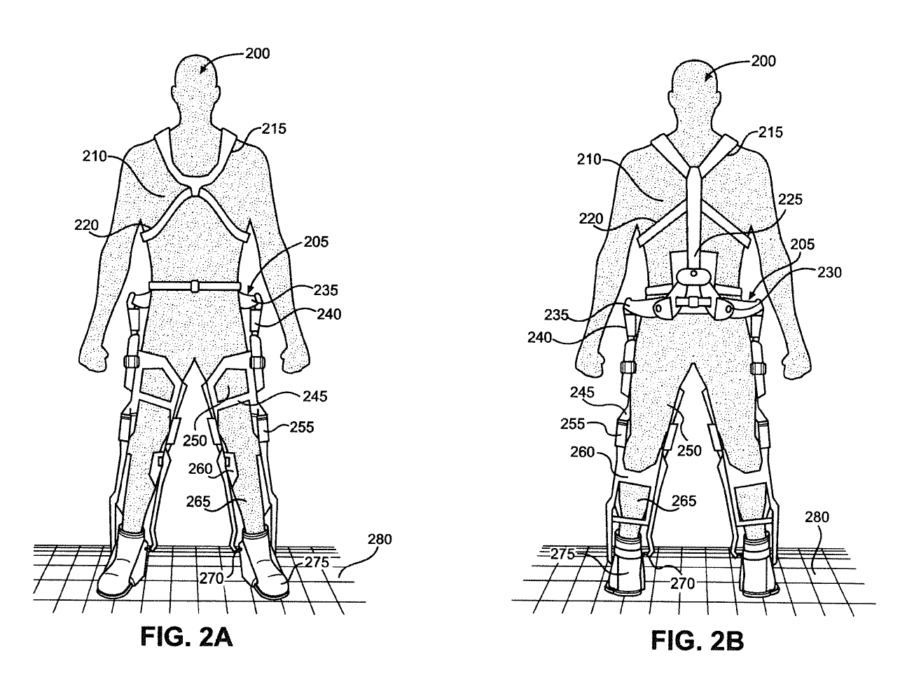

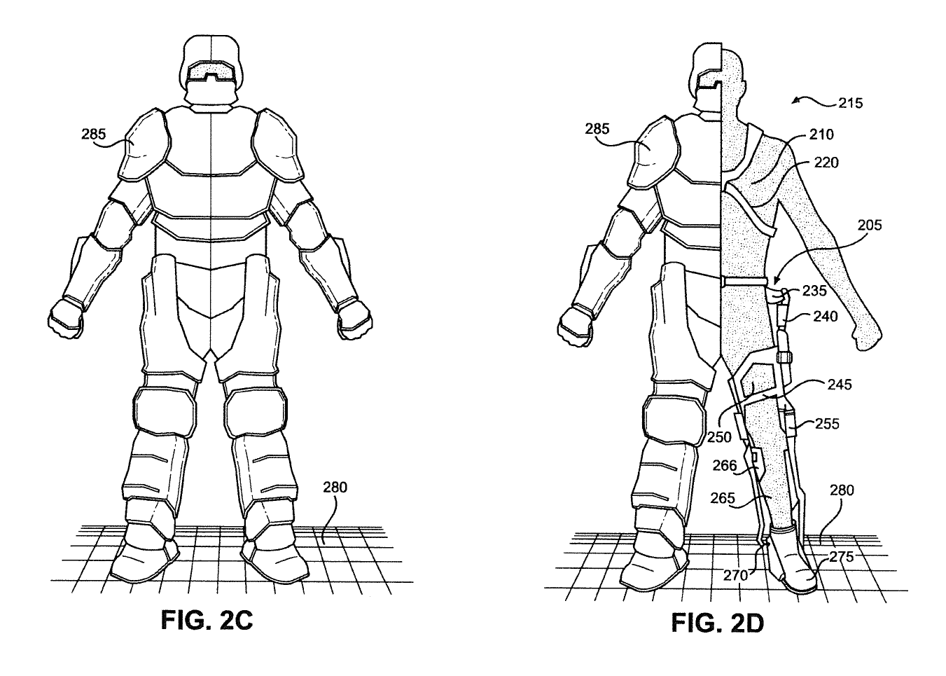

With reference to FIGS. 2A-D, a user or wearer (potentially constituted by a soldier) 200 is shown wearing an exoskeleton 205. Exoskeleton 205 is coupled to a torso 210 of user 200 by a harness 215 and strapping 220. Harness 215 is connected to a back support 225, and back support 225 is connected to a hip support 230. Hip support 230 is connected to a hip joint 235, and hip joint 235 is connected to an upper leg support 240. Upper leg support 240 is connected to an upper leg brace 245, which is coupled to an upper leg 250 of user 200. Upper leg brace 245 is connected to a knee joint 255, and knee joint 255 is connected to a lower leg brace 260. Lower leg brace 260 is coupled to a lower leg 265 of user 200 and connected to an ankle joint 270. Ankle joint 270 is connected to a foot support 275, which interacts with a surface 280 (e.g., the floor or ground). Armor 285 surrounds and is connected to exoskeleton 205, which supports the weight of armor 285. Specifically, the weight of armor 285 is transferred to surface 280 through harness 215, back support 225, hip support 230, hip joint 235, upper leg support 240, upper leg brace 245, knee joint 255, lower leg brace 260, ankle joint 270 and foot support 275. As certain particular structure of an exoskeleton for use in connection with the present invention can take various forms and is known in the art, it will not be detailed further herein.

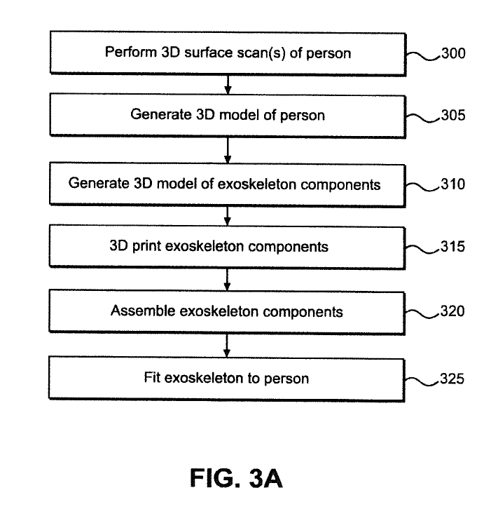

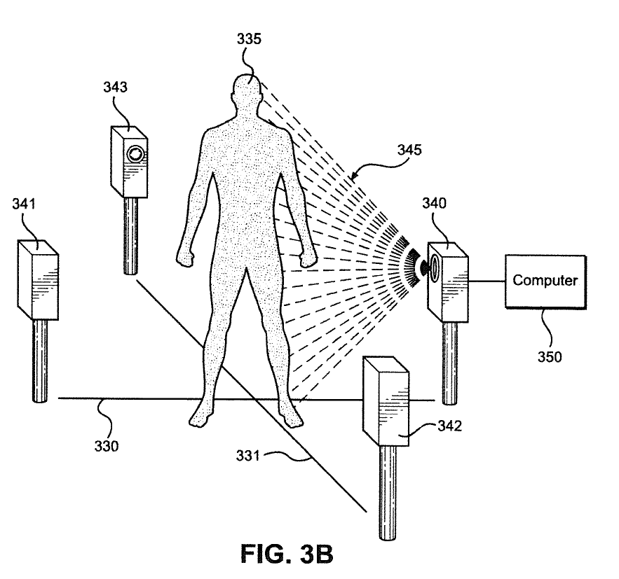

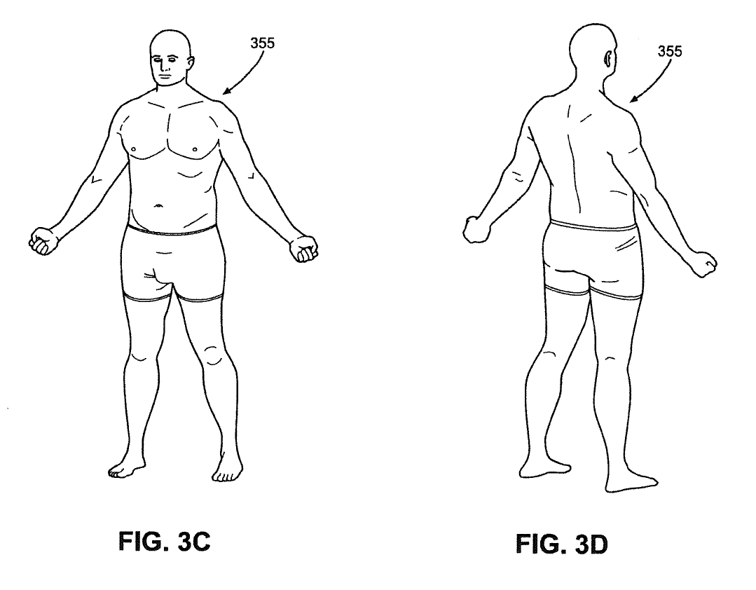

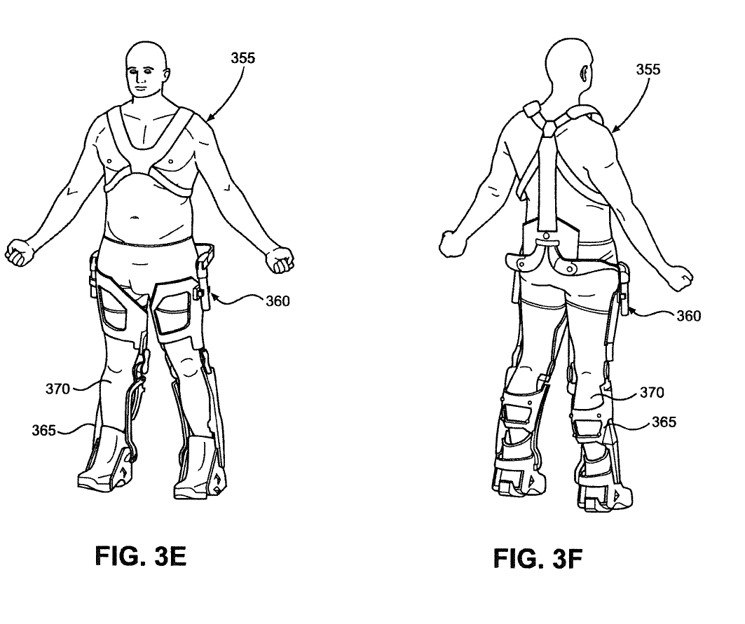

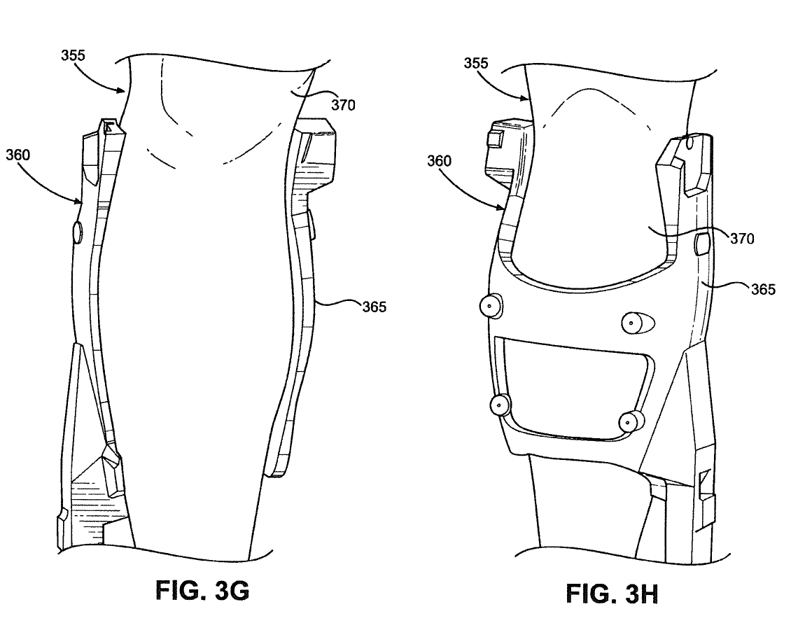

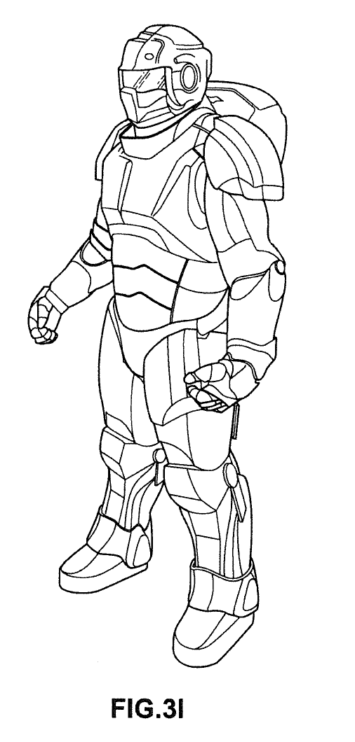

Turning to FIG. 3A, there is shown a flow chart illustrating a method in accordance with a first embodiment of the present invention. At step 300, one or more 3D scans of a person are performed in which the surface contours of the person are measured. At step 305, the 3D scan data from step 300 is used to generate a 3D surface computer model of the person. At step 310, the 3D surface model of the person is used to generate a 3D exoskeleton components model that will optimally fit the 3D surface model of the person. At step 315, 3D printing is used to fabricate exoskeleton components based on the 3D exoskeleton model generated in step 310. At step 320, a technician or physical therapist assembles the 3D printed exoskeleton components into an exoskeleton. At step 325, a technician or physical therapist fits the assembled exoskeleton to the person measured in step 300, confirms proper fit and makes further adjustments as needed.

With reference to FIG. 3B, a 3D surface scan of a person in accordance with the first embodiment is shown. Reference numerals 330 and 331 indicate a coronal plane and a sagittal plane, respectively, of a person 335. 3D scanners 340 and 341 are located along coronal plane 330, while 3D scanners 342 and 343 are located along sagittal plane 316. This allows scanners 340-343 to image person 335 from perspectives in both coronal plane 330 and sagittal plane 331. FIG. 3B shows scanner 340 emitting scanning beams 345, which interact with the surface of person 335 in such a way as to measure the 3D surface contours of person 335. Scanner 340 then transfers the data obtained from the interaction of beams 345 with person 335 to a computer (or controller or control system) 350, which stores the measurement data.

With reference now to FIGS. 3C and 3D, an exemplary 3D surface model 355 of a person in accordance with the first embodiment is shown. Surface model 355 was created by a computer using 3D laser surface scanning data resulting from a 3D surface scan of the person, using methods known to those skilled in the art of 3D surface mapping. Surface model 355 is shown from a front view in FIG. 3C and a rear view in FIG. 3D.

With reference to FIGS. 3E-I, surface model 355 is shown along with a 3D model 360 of an exoskeleton, and components thereof, in accordance with the first embodiment. As above, model 360 was created by a computer, taking into account both surface model 355 and known exoskeleton parameters (including those described in previous applications) as well as methods known in the art of 3D surface modeling. Surface models 355 and 360 are shown from a front view in FIG. 3E and from a rear view in FIG. 3F. Among other components, a lower leg brace 365 of model 360 is coupled to a right leg 370 of model 355. FIGS. 3G and 3H provide a closer view of lower leg brace 365 and right leg 370. In particular, the close fit of lower leg brace 365 to right leg 370 can be seen. Based on model 360, 3D printing was used to manufacture custom exoskeleton components, which were later fitted to the person originally modeled for the 3D scan. It was found that the custom exoskeleton pieces fit very well, allowing for a tightly-fitting, personalized exoskeleton to be assembled. This exoskeleton is shown in FIG. 3I.

As an example of the first embodiment of the present invention, consider a soldier who is about to go into a combat environment. By making use of the present invention, the soldier can be measured and modeled at a location in the United States. Upon arrival of the soldier in the theatre of combat, a custom-fitted armored exoskeleton can be 3D printed for the soldier on location using the previously generated measurements and model. If, during combat or other activities, there is damage to the soldier's exoskeleton or armor, custom-fitted replacement parts can be quickly manufactured using the previously generated models.

As a second example of the first embodiment, consider a walking-impaired patient using an ambulatory exoskeleton in a rehabilitation setting. Following certain types of injury, muscular atrophy can occur in some patients, and, over the course of rehabilitation, some regrowth of the musculature can occur. By using the present invention, a physical therapist can quickly and easily measure and model the changing physiology of the patient's legs, thereby allowing for the manufacture of better fitting exoskeleton parts so as to aid in the use of ambulatory exoskeleton therapy and the rehabilitation of the patient.

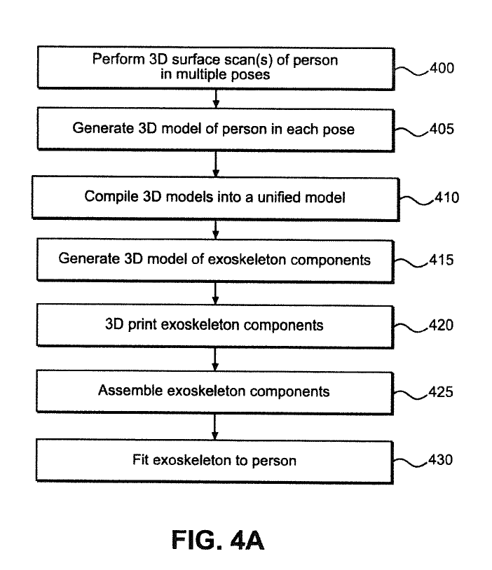

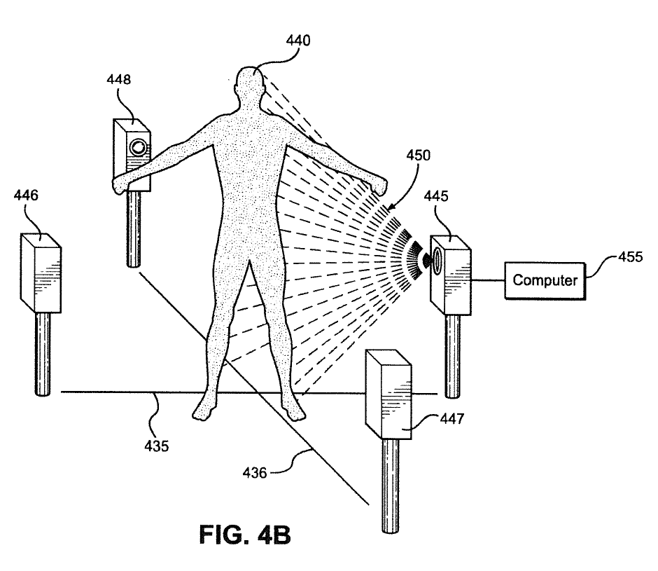

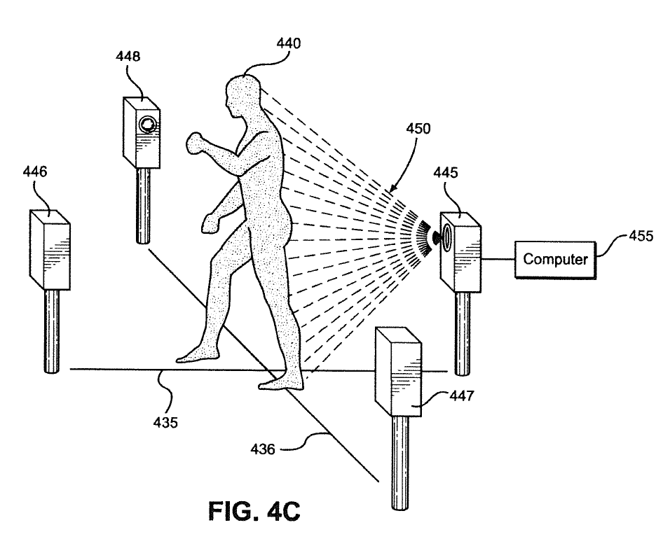

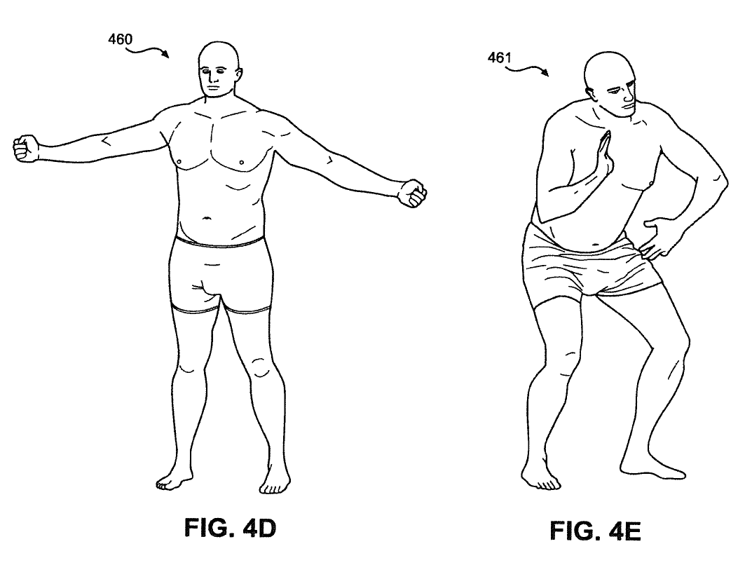

Turning to FIG. 4A, there is shown a flow chart illustrating a method in accordance with a second embodiment of the present invention. At step 400, one or more 3D scans of a person are performed for each of a plurality of poses. As a result, the surface contours of the person are measured in each of the poses. Since muscles and other tissues swell with contraction, the 3D surface of the person changes as the body of a person assumes the various poses. At step 405, the 3D scan data from step 400 is used to generate a 3D surface computer model of the person for each pose. At step 410, the 3D surface models of the person are compiled into a single, unified 3D surface model that takes into account the changing surface contours of the person in the various poses. At step 415, the unified 3D surface model is used to generate a 3D exoskeleton components model that will optimally fit the unified 3D surface model of the person. At step 420, 3D printing is used to fabricate exoskeleton components based on the 3D exoskeleton model generated in step 415. At step 425, a technician or physical therapist assembles the 3D printed exoskeleton components into an exoskeleton. At step 430, a technician or physical therapist fits the assembled exoskeleton to the person measured in step 400, confirms proper fit and makes further adjustments as needed. In some embodiments, an algorithm uses the unified model of the person to predict the position of the person's joints, allowing for modifications to the exoskeleton model to better suit the movements of the exoskeleton wearer.

With reference to FIGS. 4B and 4C, a 3D surface scan of a person in accordance with the second embodiment is shown. As with the first embodiment, reference numerals 435 and 436 indicate a coronal plane and a sagittal plane, respectively, of person 440. 3D scanners 445 and 446 are located along coronal plane 435, while 3D scanners 447 and 448 are located along sagittal plane 436. Scanner 445 is shown emitting scanning beams 450, which interact with the surface of person 440 in such a way as to measure the 3D surface contours of person 440. Scanner 445 then transfers the data obtained from the interaction of beams 450 with person 440 to a computer (or controller or control system) 455, which stores the measurement data. In contrast to the first embodiment, person 440 is scanned in each of a plurality of poses with two such poses shown in FIGS. 4B and 4C.

With reference to FIGS. 4D and 4E, exemplary 3D surface models 460 and 461 of a person in accordance with the second embodiment are shown. Surface models 460 and 461 were created by a computer using 3D laser surface scanning data resulting from 3D surface scans of the person in two different poses, using methods known to those skilled in the art of 3D surface mapping. Surface model 460 corresponds to a first pose, while surface model 461 corresponds to a second pose. The differing 3D contours of 3D surface models 460 and 461 are taken into account when a unified 3D surface model is compiled and, as a result, when the personalized exoskeleton model is designed (as described above in connection with FIG. 4A). In some embodiments, many 3D models, corresponding to various different poses, are used to create the unified model, e.g., 3 or more models can be used. Also, in some embodiments, the unified model is a moving model that can include specific actions such as walking, running or use of the arms to perform certain tasks.

As an example of the second embodiment of the present invention, consider the design of a personalized armored exoskeleton for a soldier who is highly muscular. As the bodies of different individuals develop differently with respect to physiology and physical fitness practices, the 3D surface of an individual in a single pose may not provide enough information about that individual to design an exoskeleton that fits optimally and, more importantly, moves well when being worn by that individual. By making use of the present invention, the soldier can be measured in multiple poses and modeled in such a way as to take into account muscular flex and swelling for fit of certain components and allow for significantly improved joint movement prediction for proper design of other exoskeleton components. This allows soldiers of differing physiologies to be readily measured and modeled for personalized exoskeleton design and manufacture. If, during combat or other activities, there is damage to the soldier's personalized exoskeleton or armor, custom-fitted replacement parts can be quickly manufactured using the previously generated models.

As a second example of the second embodiment of the present invention, consider a walking-impaired patient using an ambulatory exoskeleton in a rehabilitation setting. Following certain types of injury, muscular atrophy can occur in some patients, and, over the course of rehabilitation, some regrowth of the musculature can occur. Similarly, certain types of injury can prevent a patient from being able to flex certain muscles. These variations in patient physiology not only make it difficult to correctly fit a personalized exoskeleton but also complicate the use of an exoskeleton in therapy, as small variations in joint physiology can affect many activities, such as walking. By using the present invention, a physical therapist can measure the specific physiology and flex characteristics of a patient's body, allowing for the manufacture of better fitting exoskeleton parts so as to aid in the use of ambulatory exoskeleton therapy and the rehabilitation of the patient.

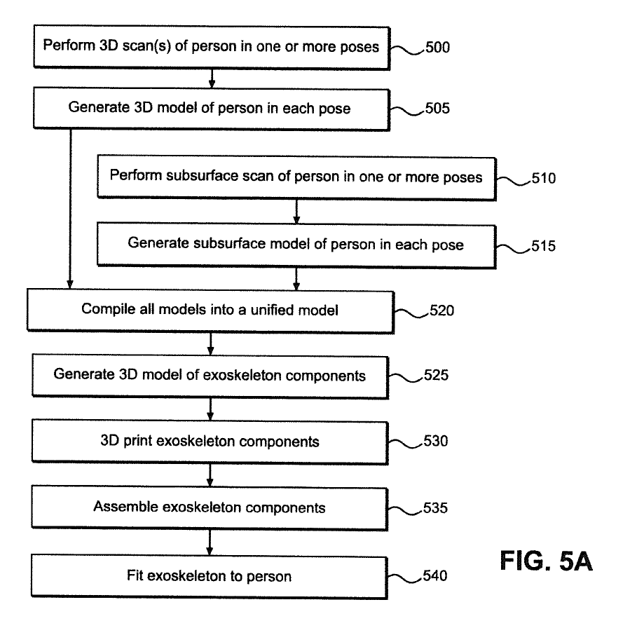

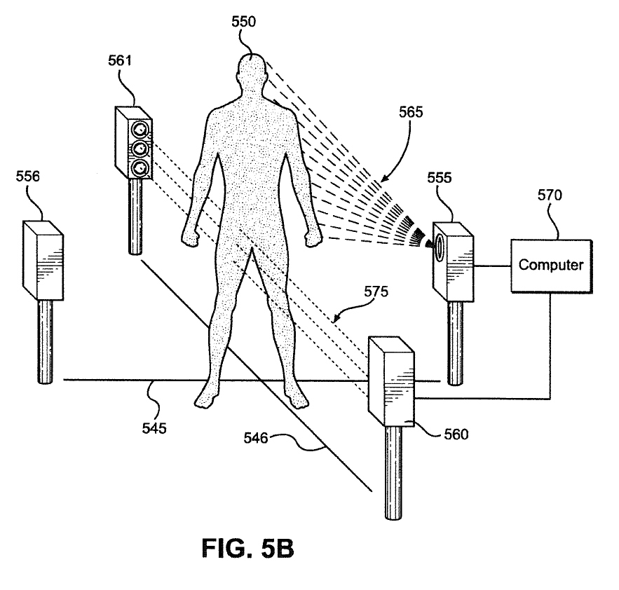

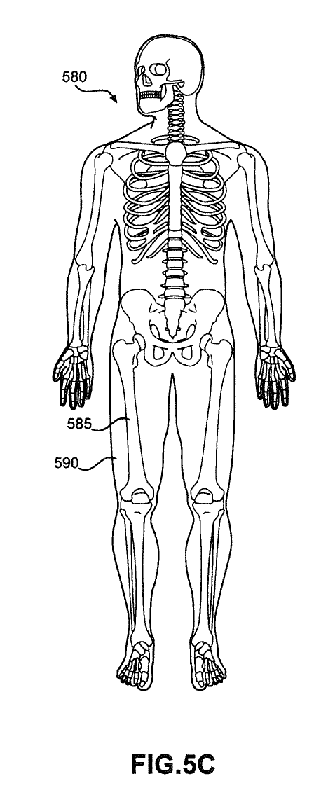

Turning to FIG. 5A, there is shown a flow chart illustrating a method in accordance with a third embodiment of the present invention. At step 500, one or more 3D surface scans of a person are performed with the person in one or more poses. At step 505, the 3D scan data from step 500 is used to generate one or more 3D surface computer models of the person. At step 510, one or more subsurface scans of the person are performed with the person in one or more poses. At step 515, the subsurface scan data from step 510 is used to create one or more subsurface models of the person. At step 520, the one or more 3D surface models and the one or more subsurface models are compiled into a single, unified model of the person that takes into account both surface and subsurface features of the person in the one or more poses. At step 525, the unified 3D model generated in step 520 is used to generate a 3D exoskeleton components model that will optimally fit the unified 3D model of the person. At step 530, 3D printing is used to fabricate exoskeleton components based on the 3D exoskeleton model generated in step 525. At step 535, a technician or physical therapist assembles the 3D printed exoskeleton components into an exoskeleton. At step 540, a technician or physical therapist fits the assembled exoskeleton to the person measured in step 500, confirms proper fit and makes further adjustments as needed. In some embodiments, an algorithm uses the unified model of the person to assign the position of the joints of the person, allowing for modifications to the exoskeleton model to better suit the movements of the exoskeleton wearer.

With reference to FIG. 5B, a 3D surface and subsurface scan of a person in accordance with the third embodiment is shown. As with the first and second embodiments, reference numerals 545 and 546 indicate a coronal plane and a sagittal plane, respectively, of person 550. 3D scanners 555 and 556 are located along coronal plane 545, while subsurface scanners 560 and 561 are located along sagittal plane 546. 3D scanner 555 is shown emitting scanning beams 565, which interact with the surface of person 550 in such a way as to measure the 3D surface contours of person 550. 3D scanner 555 then transfers the data obtained from the interaction of beams 565 with person 550 to a computer (or controller or control system) 570, which stores the measurement data. Similarly, subsurface scanner 560 is shown emitting beams 575 that penetrate and interact with the subsurface features of person 550 before being received and detected by subsurface scanner 561, at which point the signal detected by subsurface scanner 561 is relayed to computer 570, which stores the measurement data.

With reference to FIG. 5C, an exemplary subsurface model 580 of a person in accordance with the third embodiment is shown. Subsurface model 580 was created by a computer using surface scanning and subsurface scanning data resulting from 3D surface and subsurface scans of the person, using methods know to those skilled in the art of 3D surface mapping and medical imaging. Model 580 is shown from a front view front with both bones and soft tissue visible. In particular, a femur 585 and thigh tissue 590 are shown, representing bones and soft tissue, respectively. Both the surface and subsurface features of a unified model are taken into account when designing the personalized exoskeleton model (as described in connection with FIG. 5A). In some embodiments, many 3D models, corresponding to various different poses, are used to create the unified model, e.g., 3 or more models can be used. Also, in some embodiments, the unified model is a moving model that can include specific actions such as walking, running or use of the arms to perform certain tasks.

As an example of the third embodiment of this invention, consider the design of a personalized armored exoskeleton for a soldier who is highly muscular. As the bodies of different individuals develop differently with respect to physiology and physical fitness practices, the 3D surface of an individual may not provide enough information about that individual to design an exoskeleton that fits optimally and, more importantly, moves well when being worn by that individual. By making use of the present invention, both the 3D surface and the subsurface of the soldier can be measured to allow for significantly improved joint movement prediction for proper design of other exoskeleton components. This allows soldiers of different physiologies to be readily measured and modeled for personalized exoskeleton design and manufacture. If, during combat or other activities, there is damage to the soldier's personalized exoskeleton or armor, custom-fitted replacement parts can be quickly manufactured using the previously generated models.

As a second example of the third embodiment of the present invention, consider a walking-impaired patient using an ambulatory exoskeleton in a rehabilitation setting. Following certain types of injury, muscular atrophy can occur in some patients, and, over the course of rehabilitation, some regrowth of the musculature can occur. Similarly, certain types of injury can prevent a patient from being able to flex certain muscles. These variations in patient physiology not only make it difficult to correctly fit a personalized exoskeleton but also complicate the use of an exoskeleton in therapy, as small variations in joint physiology are important in many activities, such as walking. By using the present invention, a physical therapist can measure the specific physiology of a patient's body, allowing for the manufacture of better fitting exoskeleton parts so as to aid in the use of ambulatory exoskeleton therapy and the rehabilitation of the patient.

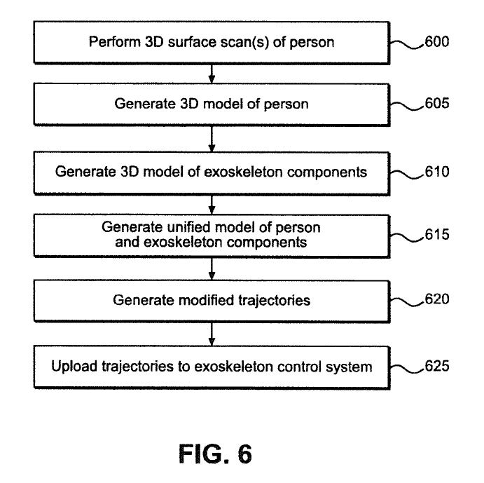

With reference to FIG. 6, there is shown a flow chart illustrating a method in accordance with the fourth embodiment of the present invention. At step 600, one or more 3D surface scans of a person are performed to measure the surface contours of the person. At step 605, the 3D scan data from step 600 is used to generate a 3D surface computer model of the person. At step 610, the 3D surface model of the person is used to generate a 3D exoskeleton components model that will optimally fit the 3D surface model of the person. At step 615, a unified model is generated from the 3D surface model and the 3D exoskeleton model. The unified model includes estimates of the movements of both the person and exoskeleton, including the person's joint positions and modifications to exoskeleton movements appropriate for the combined movements of the person and the exoskeleton. At step 620, modified exoskeleton trajectories are generated based on the unified model in order to allow an exoskeleton control system to better control the exoskeleton in conjunction with the person. At step 625, the modified exoskeleton trajectories are uploaded into the exoskeleton control system of the exoskeleton (which was constructed as described in connection with the first embodiment). In some embodiments, the modified trajectories are further modified by a technician or physical therapist based on the specific needs of the person. In addition, it should be understood that the first and fourth embodiments can be combined such that the common steps (i.e., steps 300, 305, 310, 600, 605 and 610) are performed a single time and the remaining steps (i.e., steps 315, 320, 325, 615, 620 and 625) are all performed.

As an example of the fourth embodiment of the present invention, consider a walking-impaired patient using an ambulatory exoskeleton in a rehabilitation setting. Following certain types of injury, muscular atrophy can occur in patients, and, over the course of rehabilitation, some regrowth of the musculature can occur. By using the present invention, a physical therapist is able to, for example, quickly and easily measure and model the changing physiology of a patient's legs, which allows for the automatic design of exoskeleton trajectories better suited to the rehabilitation state of the patient, thereby aiding in the use of ambulatory exoskeleton therapy and the rehabilitation of the patient.

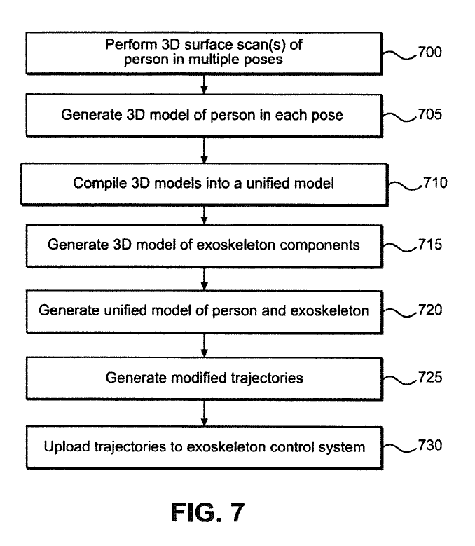

With reference to FIG. 7, there is shown a flow chart illustrating a method in accordance with the fifth embodiment of the present invention. At step 700, one or more 3D surface scans of a person are performed for each of a plurality of poses. As a result, the surface contours of the person are measured in each of the poses. Since muscles and other tissues swell with contraction, the 3D surface of the person changes as the body of a person assumes the various positions. At step 705, the 3D scan data from step 700 is used to generate a 3D surface computer model of the person for each pose. At step 710, the 3D surface models of the person are compiled into a single, unified 3D surface model that takes into account the changing surface contours of the person in the various poses. At step 715, the unified 3D surface model is used to generate a 3D exoskeleton components model that will optimally fit the 3D surface model of the person. At step 720, a unified model is generated from the 3D surface model and the 3D exoskeleton model. The unified model includes estimates of the movements of both the person and exoskeleton, including the person's joint positions, the person's surface contour changes in the various poses and modifications to exoskeleton movements appropriate for the combined movements of the person and the exoskeleton. At step 725, modified exoskeleton trajectories are generated based on the unified model of step 720 in order to allow an exoskeleton control system to better control the exoskeleton in conjunction with the person. At step 730, the modified exoskeleton trajectories are uploaded into the exoskeleton control system of the exoskeleton (which was constructed as described in connection with the second embodiment). In some embodiments, the modified trajectories are further modified by a technician or physical therapist based on the specific needs of the person. In addition, it should be understood that the second and fifth embodiments can be combined such that the common steps (i.e., steps 400, 405, 410, 415, 700, 705, 710 and 715) are performed a single time and the remaining steps (i.e., steps 420, 425, 430, 720, 725 and 730) are all performed.

As an example of the fifth embodiment of the present invention, consider a walking-impaired patient using an ambulatory exoskeleton in a rehabilitation setting. Following certain types of injury, muscular atrophy can occur in patients, and, over the course of rehabilitation, some regrowth of the musculature can occur. By using the present invention, a physical therapist is able to, for example, quickly and easily measure and model the changing physiology or strength in a patient's legs (e.g., based on muscle swell from the multiple pose surface analysis), which allows for the design of exoskeleton trajectories better suited to the rehabilitation state of the patient, thereby aiding in the use of ambulatory exoskeleton therapy and the rehabilitation of the patient.

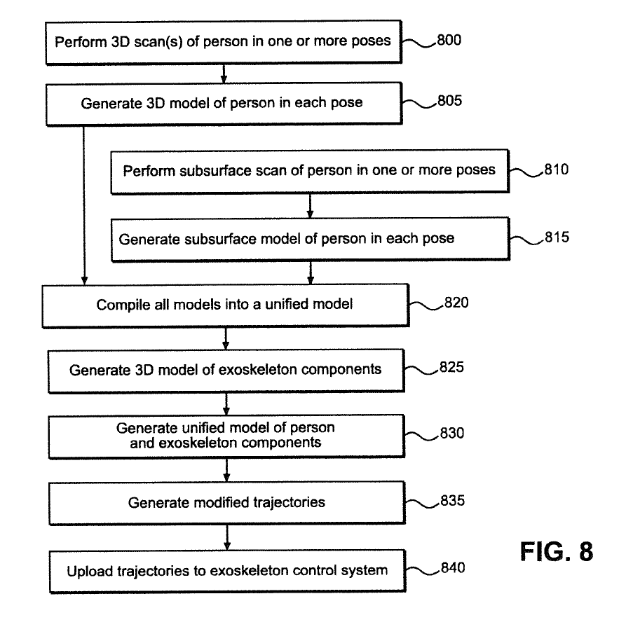

With reference to FIG. 8, there is shown a flow chart illustrating a method in accordance with the sixth embodiment of the present invention. At step 800, one or more 3D surface scans of a person are performed with the person in one or more poses. At step 805, the 3D scan data from step 800 is used to generate one or more 3D surface computer models of the person. At step 810, one or more subsurface scans of the person are performed with the person in one or more poses. At step 815, the subsurface scan data from step 810 is used to create one or more subsurface models of the person. At step 820, the one or more 3D surface models and the one or more subsurface models are compiled into a single, unified model of the person that takes into account both surface and subsurface features of the person in the one or more poses. At step 825, the unified 3D model generated in step 820 is used to generate a 3D exoskeleton components model that will optimally fit the unified 3D model of the person. At step 830, a unified model is generated from the unified model of the person generated in step 820 and the 3D exoskeleton model generated in step 825. The unified model of step 830 includes estimates of the movements of both the person and exoskeleton, including the person's joint positions, the person's surface and subsurface contours in the various poses and modifications to exoskeleton movements appropriate for the combined movements of the person and the exoskeleton. At step 835, modified exoskeleton trajectories are generated based on the unified model of step 830 in order to allow an exoskeleton control system to better control the exoskeleton in conjunction with the person. At step 840, the modified exoskeleton trajectories are uploaded into the exoskeleton control system of the exoskeleton (which was constructed as described in connection with the third embodiment). In some embodiments, the modified trajectories for the exoskeleton are further modified by a technician or physical therapist based on the specific needs of the person. In addition, it should be understood that the third and sixth embodiments can be combined such that the common steps (i.e., steps 500, 505, 510, 515, 520, 525, 800, 805, 810, 815, 820 and 825) are performed a single time and the remaining steps (i.e., steps 530, 535, 540, 830, 835 and 840) are all performed.

As example of the sixth embodiment of the present invention, consider a walking-impaired patient using an ambulatory exoskeleton in a rehabilitation setting. Following certain types of injury, muscular atrophy can occur in patients, and, over the course of rehabilitation, some regrowth of the musculature can occur. By using the present invention, a physical therapist is able to, for example, quickly and easily measure and model the changing physiology in a patient's legs based on surface and subsurface scan modeling and analysis, which allows for the design of exoskeleton trajectories better suited to the rehabilitation state of a specific patient, thereby aiding in the use of ambulatory exoskeleton therapy and the rehabilitation of the patient.

In some embodiments, all components of the exoskeleton are 3D printed based on the 3D model of the wearer and the 3D model of the exoskeleton. In other embodiments, only certain components of the exoskeleton are 3D printed based on 3D modeling of the wearer and exoskeleton, and some standard (i.e., non-custom-fit) components are assembled along with the custom components. Therefore, the three-dimensional model could be developed in various ways, including generating the three-dimensional exoskeleton model from a three-dimensional model of a non-custom-fit exoskeleton, followed by assembling the custom-fit exoskeleton by coupling the at least one three-dimensional exoskeleton component to a second non-custom-fit exoskeleton component. In some embodiments, the 3D scan, subsurface scan, 3D modeling, 3D printing and assembly take place at the same location. In other embodiments, the 3D scan, subsurface scan, 3D modeling, 3D printing and assembly take place at different locations. In some embodiments, the 3D modeling data is stored so as to allow replacement parts to be 3D printed at a later time or at a different location, e.g., the replacement parts can be printed in a local hospital or in a combat theatre/environment after initial measurements were taken elsewhere. In some embodiments, the 3D model of the person includes estimates as to the locations of the person's joints, and this information is taken into account when designing the 3D model of the exoskeleton. In some embodiments, the exoskeleton is a powered exoskeleton with actuators controlled by an exoskeleton control system, while, in other embodiments, the exoskeleton is a passive exoskeleton.

In some embodiments, all of 3D and subsurface scanners shown are used to measure the person, each of scanners being directly or indirectly in communication with the computer. Alternatively, fewer scanners are used. For example, a single 3D and/or subsurface scanner can be provider, or a single 3D and/or subsurface scanner can be provided in each of the coronal and sagittal planes. In some embodiments, a single scanner is mounted on a movable system that allows the scanner to scan from multiple angles. In other embodiments, the person stands on a rotatable platform, which allows a single scanner to image the person from multiple angles. In some embodiments, the scanners include motors so that the angles of the beams directed from the scanners can move in multiple planes. Also, in some embodiments, the scanners are arrayed in different positions than those shown in the figures. In some embodiments, multiple scans are performed concurrently, while, in other embodiments, scans are performed sequentially. In some embodiments, for example when the person is disabled, a harness or other support structure can be employed to support the person in a standing or other position.

In some embodiments, the 3D scanners are 3D laser-scanning devices. In other embodiments, the 3D scanners make use of other 3D surface measurement devices and methods known in the art of 3D surface measurement. In some embodiments, the subsurface scan makes use of a 3D surface scan, including but not limited to one or more additional 3D laser surface scans that are performed while pressurized air is simultaneously blown upon the area being scanned. The exposure to air pressure results in temporary displacement of softer tissues allowing a measurement of “soft” displaceable tissue and “hard” non-displaceable tissue. The 3D subsurface models comprises: 1) a difference map of the one or more 3D surface scans performed without pressurized air compared to the one or more 3D surface scans performed with pressurized air; or 2) simply, the one or more 3D surface scans performed with pressurized air. In some embodiments, the subsurface scan is a 3D scan that makes use of penetrating electromagnetic scanning techniques, such as a computerized tomography (CT) scan, a magnetic resonance imaging (MRI) or other 3D subsurface measurement devices and methods known in the art of medical imaging. In some embodiments, the 3D surface and subsurface scans are performed simultaneously (i.e., with one scanner type) and make use of a penetrating electromagnetic scanning technique. In some embodiments, the subsurface scan is a 2D scan that makes use of penetrating electromagnetic radiation, including but not limited to a single X-ray, with the X-ray then being processed by an algorithm that may or may not take into account the 3D surface scan data to extrapolate the 3D subsurface features of the person.

Based on the above, it should be readily apparent that the present invention provides for simple, rapid and accurate measurement of an exoskeleton user in order to allow for the subsequent design and manufacture of a personalized exoskeleton fitted to the specific user. In addition, the present invention provides for the modeling of exoskeleton and user movements for such a personalized exoskeleton in order to allow for the subsequent alteration of trajectories prescribed by an exoskeleton control system of the personalized exoskeleton. Although described with reference to preferred embodiments, it should be readily understood that various changes or modifications could be made to the invention without departing from the spirit thereof. In general, the invention is only intended to be limited by the scope of the following claims.

1. A method of creating a custom-fit exoskeleton comprising:
performing a three-dimensional surface scan of an exoskeleton wearer to generate three-dimensional surface scan data;
generating a three-dimensional surface model of the exoskeleton wearer from the three-dimensional surface scan data; and
generating a three-dimensional exoskeleton model from the three-dimensional surface model, wherein generating the three-dimensional exoskeleton model includes generating the three-dimensional exoskeleton model from a three-dimensional model of a non-custom-fit exoskeleton;
producing at least one three-dimensional exoskeleton component from the three-dimensional exoskeleton model; and
assembling the custom-fit exoskeleton by coupling the at least one three-dimensional exoskeleton component to a second non-custom-fit exoskeleton component.

2. The method of claim 1, wherein:
generating the three-dimensional surface model includes estimating a position of at least one joint of the exoskeleton wearer; and
generating the three-dimensional exoskeleton model includes generating the three-dimensional exoskeleton model using the position of the at least one joint.

3. The method of claim 1, wherein performing the three-dimensional surface scan includes performing a three-dimensional surface scan of the exoskeleton wearer in each of a plurality of poses, and generating the three-dimensional surface model includes generating a three-dimensional surface model of the exoskeleton wearer for each of the plurality of poses, the method further comprising:
compiling the three-dimensional surface models into a unified three-dimensional surface model of the exoskeleton wearer wherein generating the three-dimensional exoskeleton model includes generating the three-dimensional exoskeleton model from the unified three-dimensional surface model.

4. The method of claim 1, further comprising:
performing a subsurface scan of the exoskeleton wearer to generate subsurface scan data;
generating a subsurface model of the exoskeleton wearer from the subsurface scan data; and
compiling the three-dimensional surface model and the subsurface model into a unified model wherein generating the three-dimensional exoskeleton model includes generating the three-dimensional exoskeleton model from the unified model.

5. The method of claim 1, further comprising:
generating a unified model from the three-dimensional surface model and the three-dimensional exoskeleton model; and
generating at least one modified exoskeleton trajectory using the unified model.

6. The method of claim 5, further comprising:
uploading the at least one modified exoskeleton trajectory to an exoskeleton control system of the custom-fit exoskeleton.

7. The method of claim 1, wherein producing the printing at least one three-dimensional exoskeleton component includes printing the three dimensional exoskeleton component with a three-dimensional printer

8. The method of claim 1, further comprising:
assembling the custom-fit exoskeleton using the at least one three-dimensional exoskeleton component.

9. The method of claim 8, wherein assembling the custom-fit exoskeleton includes coupling the at least one-three dimensional exoskeleton component to a third exoskeleton component.

10. (canceled)

11. A system for creating a custom-fit exoskeleton comprising:
a three-dimensional scanner configured to perform a three-dimensional surface scan of an exoskeleton wearer to generate three-dimensional surface scan data;
at least one computer, the at least one computer being configured to:
generate a three-dimensional surface model of the exoskeleton wearer from the three-dimensional surface scan data; and
generate a three-dimensional exoskeleton model from the three-dimensional surface model; and
a three dimensional printer configured to print at least one-three dimensional exoskeleton component from the three-dimensional exoskeleton model, wherein the custom-fit exoskeleton is assembled using the at least one three-dimensional exoskeleton component.

12. The system of claim 11, wherein the at least one computer is further configured to:
estimate a position of at least one joint of the exoskeleton wearer when generating the three-dimensional surface model; and
generate the three-dimensional exoskeleton model using the position of the at least one joint.

13. The system of claim 11, wherein:
the three-dimensional scanner is further configured to perform a three-dimensional surface scan of the exoskeleton wearer in each of a plurality of poses; and
the at least one computer is further configured to:
generate a three-dimensional surface model of the exoskeleton wearer for each of the plurality of poses;
compile the three-dimensional surface models into a unified three-dimensional surface model of the exoskeleton wearer; and
generate the three-dimensional exoskeleton model from the unified three-dimensional surface model.

14. The system of claim 11, further comprising:
a subsurface scanner configured to perform a subsurface scan of the exoskeleton wearer to generate subsurface scan data, wherein the at least one computer is further configured to:
generate a subsurface model of the exoskeleton wearer from the subsurface scan data;
compile the three-dimensional surface model and the subsurface model into a unified model; and
generate the three-dimensional exoskeleton model from the unified model.

15. The system of claim 11, wherein the at least one computer is further configured to:
generate a unified model from the three-dimensional surface model and the three-dimensional exoskeleton model; and
generate at least one modified exoskeleton trajectory using the unified model.

16. The system of claim 15, wherein:
the custom-fit exoskeleton includes an exoskeleton control system; and
the at least one computer is further configured to upload the at least one modified exoskeleton trajectory to the exoskeleton control system.

17. An exoskeleton configured to be coupled to a person, the exoskeleton comprising:
a lower leg brace configured to be coupled to a lower leg of the person;
an upper leg brace configured to be coupled to an upper leg of the person;
a knee joint connected to the lower leg brace and the upper leg brace, the knee joint configured to allow relative movement between the lower leg brace and the upper leg brace;
an upper leg support connected to the upper leg brace;
a hip support; and
a hip joint connected to the upper leg support and the hip support, the hip joint configured to allow relative movement between upper leg support and the hip support,
wherein at least one of the lower leg brace, the upper leg brace, the upper leg support and the hip support is a custom-fit exoskeleton component produced from a three-dimensional exoskeleton model, the three-dimensional exoskeleton model having been generated from a three-dimensional surface model of the person and wherein the custom-fit exoskeleton component is configured to be coupled to a non-custom-fit exoskeleton component.

18. (canceled)

19. The exoskeleton of claim 17, wherein at least two of the lower leg brace, the upper leg brace, the upper leg support and the hip support are custom-fit exoskeleton components produced from the three-dimensional exoskeleton model.

20. The exoskeleton of claim 19, wherein at least one of the custom-fit exoskeleton components is configured to be coupled to a second non-custom-fit exoskeleton component.
